(19) United States
(12) Patent Application Publication
(10) Pub. No.: US 2021/0074412 A1
(43) Pub. Date: Mar. 11, 2021

(54) ADMINISTRATION OF REGULATED SAFETY REQUIREMENTS FOR HEALTH CARE PROFESSIONALS

(71) Applicant: RPHARMY, LLC, Austin, TX (US)

(72) Inventors: John PAXTON, Austin, TX (US); Laura PAXTON, Austin, TX (US)

(73) Assignee: RPHARMY, LLC, AUSTIN, TX (US)

(21) Appl. No.: 16/565,455

(22) Filed: Sep. 9, 2019

(51) Int. Cl. G16H 40/20; G16H 70/20; G16H 40/63; G16H 20/10

(52) U.S. Cl. CPC G16H 40/20; G16H 20/10; G16H 40/63; G16H 70/20

(57) ABSTRACT

Administration of regulated safety requirements for health care professionals including a medical care facility server coupled for data communications with a mobile device for use by a health care professional, the mobile device providing a safety application establishing multi-dimensional connectivity across a medical care facility. Example embodiments include a safety application receiving, from a health care professional through a mobile device, a drug inquiry including a drug ID and the safety application providing, to the health care professional in response to the drug inquiry and through the mobile device, directions including handling instructions for the identified drug.

### BACKGROUND

The United States Pharmacopeia (USP) is a pharmacopeia, that is, a compendium of drug information for the United States published annually by the United States Pharmacopeia Convention also called the “USP”. If a drug ingredient or drug product has an applicable USP quality standard (in the form of a USP-NF monograph), it must conform in order to use the designation “USP” or “NF”. Drugs subject to USP standards include both human drugs (prescription, over-the-counter, or otherwise) and also animal drugs.

USP establishes written and physical standards for medicines, food ingredients, dietary supplement products, and other products and ingredients. These standards are used by regulatory agencies and manufacturers to help to ensure that these products are of the appropriate identity, as well as strength, quality, purity, and consistency.

USP General Chapter 800 (“Chapter 800”) describes requirements for the responsibilities of health care professionals handling drugs—especially hazardous drugs. Chapter 800 also describes facility and engineering controls; procedures for deactivating, decontaminating and cleaning; spill control; and documentation of drugs. These standards apply to all healthcare personnel who receive, prepare, administer, transport or otherwise interact with hazardous drugs and all the environments in which they are handled.

Despite the requirements under Chapter 800, many health care professionals are still unaware of the risks of the drugs they are handling, administering, and supervising. As such, there is an ongoing need for methods, systems, products and other forms of administration of regulated safety requirements for health care professionals according to embodiments of the present invention described in detail below.

### BRIEF DESCRIPTION OF THE DRAWINGS

Many aspects of the present disclosure can be better understood with reference to the accompanying drawings. The components in the drawings are not necessarily to scale, with emphasis instead being placed upon clearly illustrating the principles of the disclosure. Moreover, in the drawings, like reference numerals designate corresponding parts throughout the several views. Other technical advantages may become readily apparent to one of ordinary skill in the art after review of the following figures and description.

FIG. 1 sets forth a system diagram illustrating a system for administration of regulated safety requirements for health care professionals according to example embodiments of the present invention.

FIG. 2 sets forth a flow chart illustrating a method for administration of regulated safety requirements for health care professionals according to example embodiments of the present invention.

### DETAILED DESCRIPTION

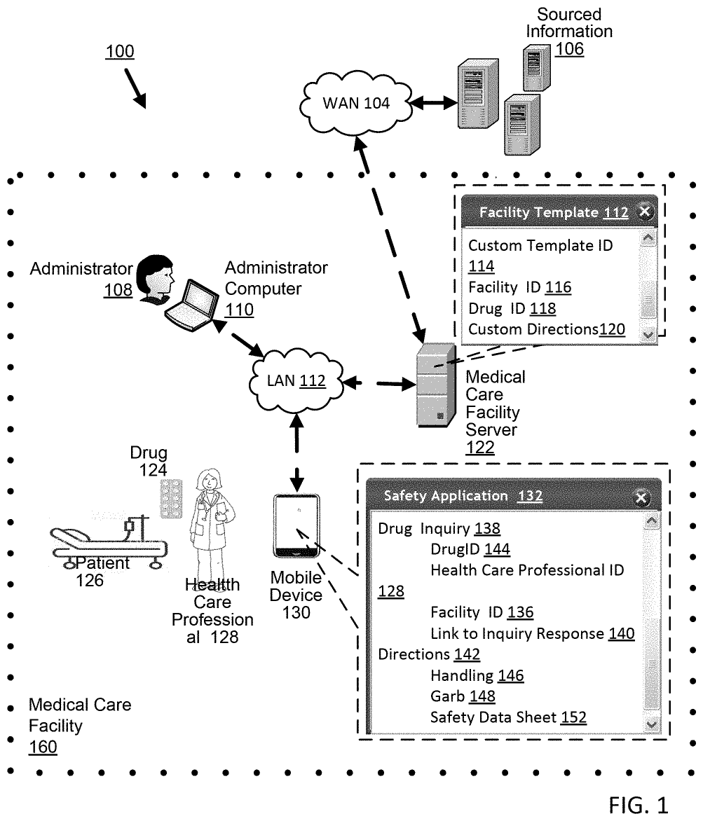

Example embodiments of the present invention are described with reference to the accompanying drawings, beginning with FIG. 1. FIG. 1 sets forth a system diagram illustrating a system for administration of regulated safety requirements for health care professionals according to example embodiments of the present invention.

In the example of FIG. 1, a medical care facility (160) provides data communications internally and externally. That is, the medical care facility (160) of FIG. 1 includes a medical care facility server (122) coupled for data communications with a mobile device (130) available to a health care professional (120) and an administrator computer (110) available to an administrator (108). In the example of FIG. 1, the medical care facility server (122), the mobile device (130), and the administrator computer (110) are coupled for data communications through a local area network (“LAN”). The example data communication is for explanation and not for limitation. In this specification, all data communications may be implemented through local area networks, wide area networks, cellular networks and so on as will occur to those of skill in the art.

In the example of FIG. 1, the devices residing in the medical care facility are also coupled for data communications with a secured information server (102) and sourced information (106). In the example of FIG. 1, the secured information server (102) and sourced information (106) are coupled for data communications with the devices in the medical care facility (160) through a wide area network (“WAN”). The example data communication is for explanation and not for limitation. As mentioned above, in this specification, all data communications may be implemented through local area networks, wide area networks, cellular networks and so on as will occur to those of skill in the art.

The example infrastructure of FIG. 1 supports administration of regulated safety requirements for health care professionals according to example embodiments of the present invention. In the example of FIG. 1, the mobile device (130) available to the health care professional (128) supports a safety application (132) to aid in safe care for the patient (126). Safe care includes safety for the health care professional (128)—not just the patient (126). Health care professionals who deserve such safety include professionals in pharmacies, veterinarian clinics, hospitals, emergent care units, rescue missions, loading docks, medical transportation, and so on as will occur to those of skill in the art.

The example illustrated and described in FIG. 1 depicts a medical care facility. This is for example and not for limitation. Administration of regulated safety requirements for health care professionals according to example embodiments of the present invention may be implemented in pharmacies, veterinarian clinics, hospitals, emergent care units, rescue missions, transportation operations, and so on as will occur to those of skill in the art.

In the example of FIG. 1, the safety application (132) receives, from a health care professional (128) through the mobile device (130), a drug inquiry (138). A drug inquiry (138) is a digital request according to embodiments of the present invention that returns to the health care professional (128) safety information directing the professional to handle the drug in a safe manner, wear the proper garb to protect the professional, and other instructions, directives, and directions for safe administration of the drug and patient.

In the example of FIG. 1, the safety application (132) provides to the health care professional (128), in response to the drug inquiry (138) and through the mobile device (130), directions (142) for guiding the health care professional (128) in safe and beneficial care of the patient (126). In the example of FIG. 1, the directions (142) include handling instructions (146) for the identified drug (144). Handling instructions are detailed directives instructing the health care professional (128) in the physical administration of the drug. Such handling instructions include how to safely store the drug, how to safely transport the drug, waste management instructions for the identified drug and so on as will occur to those of skill in the art. Such handling instructions may be presented through a user interface in the safety application (132) as will occur to those of skill in the art.

In the example of FIG. 1, the directions (142) also include garb information identifying clothing for the health care professional (128). Such garb information (148) may include advised gloves for handling the identified drug, clothes to be worn by the health care professional (128) while handling the identified drug, masks for the health care professional (128), and other garb as will occur to those of skill in the art.

In the system of FIG. 1, the safety application (132) also provides to the health care professional (128) through the mobile device (130) a safety data sheet. A safety data sheet (152) or sometimes called a material safety data sheet (MSDS), or product safety data sheet (PSDS) is a document that list information relating to occupational safety and health for the use of various substances and products. Such safety data sheets are a widely used system for cataloging information on chemicals, chemical compounds, and chemical mixtures. SDS information may include instructions for the safe use and potential hazards associated with a particular material or product, along with spill-handling procedures.

In the example of FIG. 1, the safety data sheet (152) is a dynamic record linked to real-time secured information provided by a secured information server (102). Such secured information is information provided and updated within the medical care facility (160) itself. In the example of FIG. 1, secured information may come in the form of information provided by an administrator (108) or other operative in the facility uploaded to a medical care facility server (122) through the administrator computer (110). In the example of FIG. 1, the medical care facility server (122) exposes a facility template application (122) that allows the administrator (108) to use a custom template (144) having a custom facility ID (116) and provide for any particular drug (118) in this facility custom directions (120) for the administration and regulation of that drug within the medical care facility (160). As such, the administrator (108) is empowered to customize the variables of the safety application (132) for that facility (160).

In the example of FIG. 1, the safety data sheet is a dynamic record linked to real time sourced information (106). In the example of FIG. 1, sourced information is publicly known information about a drug and its administration. Such publicly know information is dynamically obtained and in the example of FIG. 1, such dynamic information is used to update the safety application (132) such that the health care professional is given the best information possible and the best protection possible in administering drugs including updated handling, garb, and administration data and updated safety data sheets.

In the example of FIG. 1, the safety application is administered an updated by a third-party service provider using sourced and/or local information to provide the best directions for the health care professional.

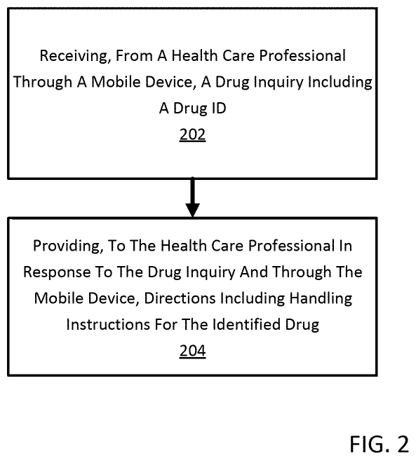

For further explanation, FIG. 2 sets forth a method for administration of regulated safety requirements for health care professionals according to example embodiments of the present invention. The method of FIG. 2 includes receiving (202), from a health care professional through a mobile device, a drug inquiry including a drug ID.

The method of FIG. 2 also includes providing (204), to the health care professional in response to the drug inquiry and through the mobile device, directions including handling instructions for the identified drug. As mentioned above, handling instructions may include storage instructions for the identified drug, transportation instructions for the identified drug, waste instructions for the identified drug and other handling instructions as will occur to those of skill in the art.

Also as mentioned above, directions may include garb information identifying clothing for the health care professional including proper gloves for handling the identified drug, clothes to be worn by health care professional while handling the identified drug and other garb that will occur to those skill in the art.

Modifications, additions, or omissions may be made to the various embodiments described herein without departing from the scope of the disclosure. For example, the components of the systems and apparatuses may be integrated or separated. Moreover, the operations of the various embodiments disclosed herein may be performed by more, fewer, or other components and the methods described may include more, fewer, or other steps. Additionally, steps may be performed in any suitable order.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is:

1. A system for administration of regulated safety requirements for health care professionals, the system comprising:
medical care facility server coupled for data communications with a mobile device for use by a health care professional, the mobile device providing a safety application establishing multi-dimensional connectivity across a medical care facility;
the safety application receiving, from a health care professional through a mobile device, a drug inquiry including a drug ID;
the safety application providing, to the health care professional in response to the drug inquiry and through the mobile device, directions including handling instructions for the identified drug.

2. The system of claim 1 wherein handling instructions include storage instructions for the identified drug.

3. The system of claim 1 wherein handling instructions include transportation instructions for the identified drug.

4. The system of claim 1 wherein handling instructions include waste instructions for the identified drug.

5. The system of claim 1 wherein directions include garb information identifying clothing for the health care professional.

6. The system of claim 5 wherein garb information includes proper gloves for handling the identified drug.

7. The system of claim 5 wherein garb information includes clothes to be worn by health care professional while handling the identified drug.

8. The system of claim 1 further comprising providing to the healthcare professional through the safety application an electronic safety data sheet.

9. The system of claim 8 wherein the safety data sheet is a dynamic record linked to real time secured information provided by a secured information server.

10. The system of claim 8 wherein the safety data sheet is a dynamic record linked to real time sourced information server.

11. A method for administration of regulated safety requirements for health care professionals, the method comprising:
receiving, from a health care professional through a mobile device, a drug inquiry including a drug ID; and
providing, to the health care professional in response to the drug inquiry and through the mobile device, directions including handling instructions for the identified drug.

12. The method of claim 11 wherein handling instructions include storage instructions for the identified drug.

13. The method of claim 11 wherein handling instructions include transportation instructions for the identified drug.

14. The method of claim 11 wherein handling instructions include waste instructions for the identified drug.

15. The method of claim 11 wherein directions include garb information identifying clothing for the health care professional.

16. The method of claim 15 wherein garb information includes proper gloves for handling the identified drug.

17. The method of claim 15 wherein garb information includes clothes to be worn by health care professional while handling the identified drug.

18. The method of claim 11 further comprising providing to the healthcare professional through the safety application an electronic safety data sheet.

19. The method of claim 18 wherein the safety data sheet is a dynamic record linked to real time secured information provided by a secured information server.

20. The method of claim 18 wherein the safety data sheet is a dynamic record linked to real time sourced information server.
